(19) United States
(12) Patent Application Publication
(10) Pub. No.: US 2020/0281640 A1
(43) Pub. Date: Sep. 10, 2020

(54) PERCUTANEOUS BIOPSY CAUTERIZATION DEVICE AND METHODS OF USE

(71) Applicants: Gurvan Edmond Blackman, Robbinsville, NJ (US); David Christopher Feldstein, Haddonfield, NJ (US)

(72) Inventors: Gurvan Edmond Blackman, Robbinsville, NJ (US); David Christopher Feldstein, Haddonfield, NJ (US)

(21) Appl. No.: 16/809,274

(22) Filed: Mar. 4, 2020

Related U.S. Application Data
(60) Provisional application No. 62/813,234, filed on Mar. 4, 2019.

(51) Int. Cl. A61B 18/08; A61B 10/04

(52) U.S. Cl. CPC A61B 18/082; A61B 2018/00595; A61B 2010/045; A61B 10/04

(57) ABSTRACT

A cautery device having a proximal end and a distal end includes a housing, a battery encased within the housing; a trigger disposed on the housing, a metallic rod in electrical communication with the battery, and an insulating jacket disposed over a portion of the metallic rod, the insulating jacket being smaller than 12 gauge.

### CROSS-REFERENCE TO RELATED APPLICATIONS

This application claims priority to U.S. Provisional Application Ser. No. 62/813,234, filed Mar. 4, 2019, entitled “PERCUTANEOUS BIOPSY CAUTERIZATION DEVICE AND METHODS OF USE,” the contents of which are fully incorporated as if fully set forth herein.

### FIELD OF THE DISCLOSURE

The present disclosure relates to biopsies in medical settings. More particularly the present disclosure relates to devices and methods to aid in cauterizing vessels after performing a percutaneous biopsy procedure.

### BACKGROUND OF THE DISCLOSURE

Certain medical procedures employ the use of needles to puncture a patient's skin. The use of such needles is typically temporary. One example of such a procedure is a percutaneous biopsy, in which a needle is passed to a target location within the body to sample tissue. After extraction, the sampled tissue is analyzed to determine if the tissue is benign, malignant or infectious. For example, biopsies may be used to diagnose lymphoma, kidney cancer, liver cancer, lung cancer, and other soft tissue cancer. A biopsy might also be needed to diagnose a variety of other conditions and/or diseases.

After a biopsy procedure is performed and the instruments are removed, there is a risk of bleeding at the site of the biopsy. This is especially true for biopsies where the target is an organ with dense vasculature.

### SUMMARY OF THE DISCLOSURE

In some embodiments, a cautery device having a proximal end and a distal end includes a housing, a battery encased within the housing, a trigger disposed on the housing, a metallic rod in electrical communication with the battery, and an insulating jacket disposed over a portion of the metallic rod.

### BRIEF DESCRIPTION OF THE DISCLOSURE

Various embodiments of the presently disclosed biopsy devices are shown herein with reference to the drawings, wherein:

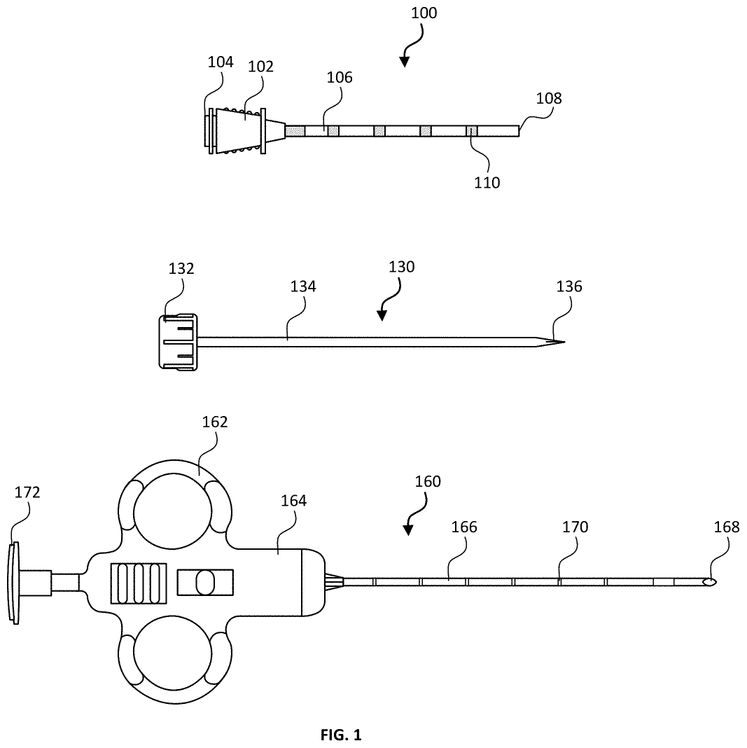

FIG. 1 is a schematic side view of a biopsy kit including an introducer, a stylet and a biopsy needle;

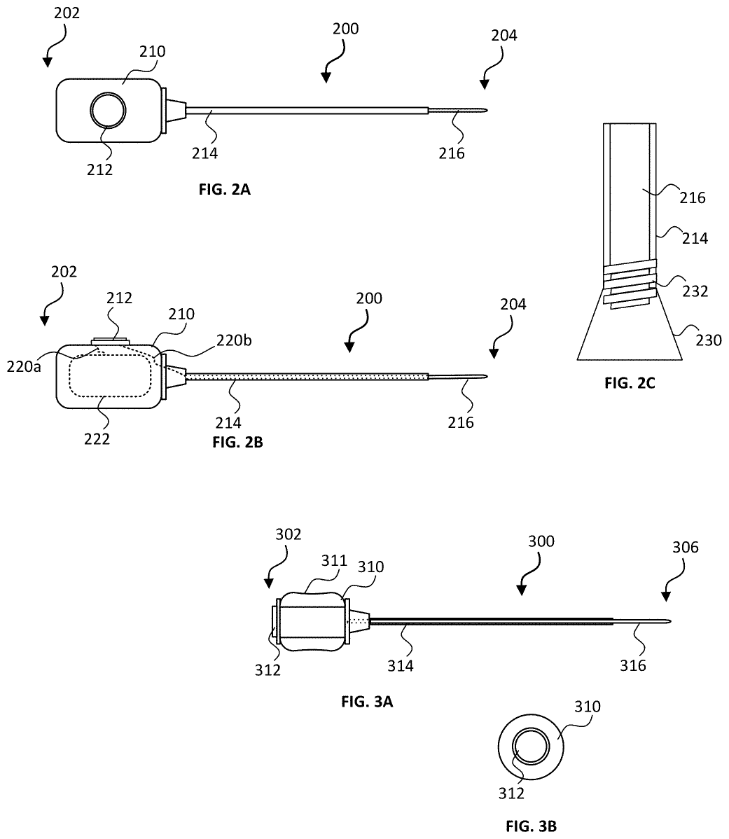

FIGS. 2A-B are schematic top and side views of one embodiment of a cautery device;

FIG. 2C is a schematic cross-sectional view of a base for coupling a metallic rod to the housing; and

FIGS. 3A-B are schematic side and rear end views of another embodiment of a cautery device.

Various embodiments of the present invention will now be described with reference to the appended drawing. It is to be appreciated that these drawings depict only some embodiments of the invention and are therefore not to be considered limiting of its scope.

### DETAILED DESCRIPTION

Despite the various improvements that have been made to biopsy devices and their methods of use, conventional devices suffer from some shortcomings as described above.

There therefore is a need for further improvements to the devices, systems, and methods of closing and sealing spaces after biopsy procedures. Among other advantages, the present disclosure may address one or more of these needs.

FIG. 1 illustrates a biopsy kit that generally includes an introducer 100, a stylet 130 and a biopsy needle 160. Each of these components will be described generally in order to appreciate the functionality of the cauterization device. Introducer 100 includes a hub 102 having an entrance port 104, and a hollow cannula 106 having a lumen 108 in communication with the entrance port. Entrance port 104 and hollow cannula 106 may be sized to accept either one of stylet 130 or biopsy needle 160. In at least some examples, introducer 100 may be 13 to 19 gauge and may be 8 cm to 23 cm in length. Optionally, introducer 100 may include depth markings 110 on cannula 106, spaced apart to allow a physician to determine the depth to which the cannula has been inserted into a patient's body. The kit may also include stylet 130, which includes hub 132 connected to metallic needle 134, the metallic needle 134 terminating in a sharp tip 136. Optionally, metallic needle 134 terminates in a blunt tip (not shown). Stylet 134 is sized to be inserted within introducer 100.

The kit also includes a biopsy needle 160. Biopsy needle 160 generally includes a handle 102, a main body 164, and a metallic inner core (not shown) and an outer sheath 166. Handle 162 is configured to be grasped by the physician and may include a pair of rings as shown, or a pair of perpendicularly extending arms. Inner core and outer sheath 166 may include a compartment 168 for collecting a specimen during a biopsy. In one example, the inner core and the outer sheath are movable relative to one another, and firing the device causes a movement of one of the components with respect to the other adjacent body tissue, the movement causing a sample of the tissue to be collected within compartment 168.

Outer sheath 166 may also include a plurality of spaced indicator markings 170 as shown. Typical biopsy needles also include a plunger 172 or other trigger mechanism configured to allow collection of the specimen. In the example shown, biopsy needle 160 includes a plunger 172 that may be retracted and pushed to cock and fire the outer sheath to rapidly move over the inner core to collect a sample.

A typical biopsy procedure will now be briefly described to better understand the functionality of each of these components. First, the site is prepared and adequate anesthesia is provided to the patient. A scalpel may be used to puncture the skin. The introducer and stylet may be inserted into the patient to a position proximal to the lesion to be biopsied. Imaging guidance may be useful at this step. The stylet may then be removed, leaving the introducer in place as a guide for the biopsy needle. The biopsy needle may then be cocked, for example, using a plunger and an appropriate penetration depth may be chosen. After the biopsy needle is advanced to the appropriate site, it may be fired to capture a specimen. The biopsy needle may be removed and the specimen removed. This process may be repeated if additional biopsies are required.

At this juncture, it may be possible to use a cautery device as illustrated in FIGS. 2A-3B. Cautery device 200 generally extends between a proximal end 202 and a distal end 204, and includes a housing 210 having a trigger 212, and an insulating jacket 214 extending over a portion of a metallic rod (or wire) 216. Insulating jacket 214 may cover most of metallic rod 216 except for an exposed portion at the distal end. In at least some examples, exposed portion of the metallic rod 216 is in the range of 0.5 to 3.0 mm. Both insulating jacket and metallic rod 216 may be formed of a diameter that is small enough fit within the cannula of introducer 100. Introducers 100 may range from 11 gauge to 19 gauge, and biopsy needles may range from 12 to 22 gauge. In at least some examples, insulating jacket has an outer diameter of between 12 gauge and 22 gauge. In other embodiments, a cautery device 200 may be formed without the insulating jacket. In those cases, the metallic rod may be formed slightly larger than embodiments with the insulating jacket, and the metallic rod may have an outer diameter of 12 gauge to 22 gauge. In at least some examples, the largest diameter on the metallic rod and/or insulating jacket may be one gauge higher than the accompanying introducer.

Suitable materials for the housing and the insulating jacket may include a polymeric material, such as silicone, thermoplastic polyurethanes (TPU), rubber, plastic, polypropylene, polyethylene, acrylonitrile butadiene styrene (ABS), high impact polystyrene (HIPS), polyvinyl chloride (PVC) polycarbonate, thermoplastic elastomers, polybutylene terephthalate, ethylene vinyl acetate, nylon a low-density polyethylene, linear low-density polyethylene, and suitable combinations thereof. Suitable materials for the metallic rod may include copper, stainless steel or other conductive metals.

In some examples, insulating jacket 214 may be translatable relative to the metallic rod 216 so that the length of the exposed portion of the wire (i.e., the portion of the distal tip of the wire not covered by the insulating jacket) may be varied. In at least some other embodiments, the insulating jacket may be formed of two or more concentric segments that telescope relative to one another so that the length of the insulating jacket may be varied, and with it the length of the exposed portion of the metallic rod.

As shown in FIG. 2B, the generally cylindrical housing 210 encases a battery 222 coupled to trigger 212 via wire 220a, the trigger 212 being in turn coupled to metallic rod 216 via wire 220b. In one example, trigger 212 may be in the form of a button, that when pressed, electrically connects the metallic rod 216 to battery 222 and causes the metallic rod to heat up to a temperature of between 500 and 1500 degrees Fahrenheit.

In some example, the cautery device 200 is disposable and made in a cost-effective manner so that it used for a single procedure and then discarded. Alternatively, the housing and the components therein may be used. For example, battery 222 may hold sufficient charge for multiple procedures. Additionally, battery 222 may be rechargeable. In such applications, the metallic rod 216 may be connected to the housing (and the wire 220a) via a base (FIG. 2C). As shown in this example, metallic rod 216 and/or insulating jacket 214 may be releasably coupleable to the housing via, for example, a base 230 having a threaded mechanism 232. In such instances, housing and base 230 may be reused for multiple procedures while the metallic rod and/or insulating jacket may be discarded after use and replaced. Base 230 may also serve to electrically connect the metallic rod to the battery.

FIG. 3A-B illustrates another embodiment of a cautery device, the device having some additional features and/or modifications. Cautery 300 extends between a proximal end 302 and a distal end 304, and includes a housing 310 having a circumferential depression 311 for improved ergonomics. A trigger 312 is disposed on the proximal end of the device in line with the metallic 316 and the insulating jacket 314. Housing 310 may encase a battery coupled to the trigger as previously described. In one other variation, metallic rod 316 may be retractable within jacket 314 so that it is exposed during use, and capable of being totally pulled within the jacket when not in use.

In use, the physician may remove the biopsy needle after obtaining the required sample(s). With the introducer still in place, the physician may advance the cautery device within the cannula of the introducer into the site of the biopsy. When cautery device is in the proper position, the physician may actuate the trigger which causes the metallic rod to be in electrical communication with the battery, heating up the metallic rod to 200 degrees Fahrenheit. The heated wire will cauterize the surrounding tissue, sealing the area of the biopsy and preventing any excess bleeding. Insulating jacket may surround portions of the metallic rod so that only the wire's distal tip is exposed. This may prevent the remaining portions of the wire from heating up the inside of the introducer or from other tissue such as muscle tissue. The cautery device may then be removed and discarded. While a simple mechanism has been described, in some examples, the cautery device utilizes radiofrequency ablation, cryoablation, or laser ablation.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

It will be appreciated that the various dependent claims and the features set forth therein can be combined in different ways than presented in the initial claims. It will also be appreciated that the features described in connection with individual embodiments may be shared with others of the described embodiments.

What is claimed is:

1. A cautery device having a proximal end and a distal end, comprising:
a housing;
a battery encased within the housing;
a trigger disposed on the housing;
a metallic rod in electrical communication with the battery; and
an insulating jacket disposed over a portion of the metallic rod, the insulating jacket being smaller than 11 gauge.

2. The cautery device of claim 1, wherein the insulating jacket covers a majority of the metallic rod.

3. The cautery device of claim 1, wherein the insulating jacket covers a majority of the metallic rod so that only an exposed portion of the metallic rod is between 0.5 and 3.0 mm.

4. The cautery device of claim 1, wherein the insulating jacket includes a polymeric material.

5. The cautery device of claim 1, wherein the insulating jacket includes thermoplastic polyurethane.

6. The cautery device of claim 1, wherein the insulating jacket is translatable relative to the metallic rod so that an exposed length of the metallic rod is variable.

7. The cautery device of claim 1, wherein the insulating jacket includes two or more concentric segments.

8. The cautery device of claim 1, further comprising a wire in electrical communication with the trigger and the battery.

9. The cautery device of claim 1, wherein the battery is configured to heat up the metallic rod to a temperature of between 500 and 1500 degrees Fahrenheit.

10. The cautery device of claim 1, wherein the metallic rod is releasably coupleable to the housing.

11. The cautery device of claim 1, further comprising a base adjacent the housing, the base having a threaded connection to the housing.

12. A kit comprising:
an introducer;
a biopsy needle; and
the cautery device of claim 1, the insulating jacket of the cautery device being configured, arranged and sized to fit within the introducer.
